(19) United States
(12) Patent Application Publication
(10) Pub. No.: US 2025/0313896 A1
(43) Pub. Date: Oct. 9, 2025

(54) METHODS FOR EVALUATING A SUBJECT FOR FRAGILE X SYNDROME

(71) Applicant: CHILDREN'S HOSPITAL MEDICAL CENTER, Cincinnati, OH (US)

(72) Inventors: Craig Andrew ERICKSON, Cincinnati, OH (US); Lauren SCHMITT, Cincinnati, OH (US)

(21) Appl. No.: 19/172,991

(22) Filed: Apr. 8, 2025

Related U.S. Application Data
(60) Provisional application No. 63/575,926, filed on Apr. 8, 2024.

(51) Int. Cl. C12Q 1/6883; C12Q 1/6874

(52) U.S. Cl. CPC C12Q 1/6883; C12Q 1/6874; C12Q 2600/154

(57) ABSTRACT

Methods for determining methylation status and CGG repeat extent in a untranslated region (UTR) of Fragile X messenger riboprotein gene 1 (FMR1) in a subject comprise (a) isolating high molecular weight DNA from a whole blood sample; (b) enriching the isolated DNA for an UTR of FMR1 with Cas9-assisted gene-targeted cleavage of regions of interest in the FMR1 gene and ligation of sequencing adapters to the cleaved regions of interest; and (c) sequencing the regions of interest to determine a cumulative number of methylated CGG repeats in the regions of interest and a cumulative number of CGG repeats in the regions of interest. Methods for evaluating a subject for Fragile X Syndrome further comprise (d) relating the cumulative number of CGG repeats and the cumulative number of methylated CGG repeats to a clinical phenotype of Fragile X Syndrome.

### CROSS-REFERENCE TO RELATED APPLICATIONS

This application claims the benefit of the filing date of U.S. Provisional Application No. 63/575,926, filed Apr. 8, 2024, the entire contents of which are incorporated by reference herein.

### FIELD OF THE INVENTION

The present invention is directed to methods for determining methylation status and CGG repeat extent in a region of Fragile X messenger riboprotein gene 1 (FMR1) in a subject. The invention is also directed to methods for evaluating a subject for Fragile X Syndrome, and particularly for evaluating a subject for a phenotype of Fragile X Syndrome.

### BACKGROUND OF THE INVENTION

Fragile X Syndrome (FXS) is the most common inherited form of intellectual disability and the most common monogenic form of autism spectrum disorder. Although the genetic cause of FXS, a CGG trinucleotide repeat expansion in the 5′ untranslated region (UTR) of the Fragile X Messenger Riboprotein gene 1 (FMR1) leading to loss of the Fragile X Messenger Riboprotein (FMRP), was discovered more than two decades ago, many aspects of the disorder are still incompletely understood. Therefore, current treatments are mainly symptomatic, with limited efficacy for core developmental impairments associated with the disorder, and often preventing early intervention for possible treatment or accommodations.

The CGG repeat expansion in the FMR1 gene is usually categorized into “normal” (≤44 repeats), intermediate or grey zone (45-54), pre-mutation (55-200 repeats) and full mutation (>200 repeats). While premutation carriers can develop health issues such as Fragile X-associated tremor/ataxia syndrome (FXTAS), most commonly in men, and Fragile X-associated primary ovarian insufficiency (FXPOI) in women, in general, only individuals with a full mutation have gene hypermethylation, subsequent reduction in FMRP expression and FXS.

Diagnosis of FXS has conventionally been made using Southern Blot and PCR or methylation PCR technology which provide read outs as “fully methylated,” “partially methylated” or “not methylated,” with fully methylated resulting in an FXS diagnosis. Shortcomings of conventional testing are apparent however from recent showings that even “fully methylated” patients according to Southern Blot and PCR or PCR methylation, express some amount of FMRP. Additionally, even though FXS is a monogenic disorder, it is marked by considerable phenotypic variability. Some affected individuals can obtain high school and post-high school degrees, while others may remain nonverbal and severely developmentally impaired into adulthood. This variability is partially caused by the location of the FMR1 gene on the X chromosome, as phenotypic presentation in females with one fully expanded allele can significantly vary due to random X chromosome inactivation. However, even males with FXS have a wide range of phenotypes suggesting additional contributing factors.

Manifestation of phenotype symptoms and extent of disabilities often prevent early intervention for assistance and/or accommodation. Accordingly, it would be advantageous to provide a means for more accurately evaluating FXS disease phenotypes, which can be useful in assessing expected disability extent and in providing an opportunity for early assistance and/or accommodation.

### SUMMARY OF THE INVENTION

Accordingly, it is an object of the invention to provide methods which assist in evaluating FXS disease phenotypes by improved methylation and repeat count assessment.

In one embodiment, the invention is directed to a method for determining methylation status and CGG repeat extent in an untranslated region (UTR) of Fragile X messenger riboprotein gene 1 (FMR1) in a subject. The method comprises (a) isolating high molecular weight DNA from a whole blood sample from the subject; (b) enriching the isolated high molecular weight DNA for an UTR of FMR1 with Cas9-assisted gene-targeted cleavage of regions of interest in the UTR of FMR1 gene and ligation of sequencing adapters to the cleaved regions of interest; and (c) sequencing the regions of interest to determine a cumulative number of methylated CGG repeats in the regions of interest and a cumulative number of CGG repeats in the regions of interest.

In another embodiment, the invention is directed to a method for evaluating a subject for Fragile X Syndrome. The method comprises (a) isolating high molecular weight DNA from a whole blood sample from the subject; (b) enriching the isolated high molecular weight DNA for a UTR of Fragile X messenger riboprotein gene 1 (FMR1) with Cas9-assisted gene-targeted cleavage of regions of interest in the UTR of FMR1 gene and ligation of sequencing adapters to the cleaved regions of interest; (c) sequencing the regions of interest to determine a cumulative number of methylated CGG repeats in the regions of interest and a cumulative number of CGG repeats in the regions of interest; and (d) relating the cumulative number of CGG repeats and the cumulative number of methylated CGG repeats to a clinical phenotype of Fragile X Syndrome in the subject.

The methods of the invention are advantageous in evaluating FXS disease phenotypes, and are suitable for use in assessing expected long term disability and in providing an opportunity for early intervention and/or accommodation and support.

### BRIEF DESCRIPTION OF THE DRAWINGS

The detailed description will be more fully understood in view of the drawings in which:

FIG. 1 shows results of conventional Southern Blot diagnosis of six “fully methylated” male patients.

FIG. 2 shows the relation between CGG count and methylation load in male and female FXS patients;

FIGS. 3A and 3B show the relation between FMRP as measured by assay and CGG repeat count and methylation load, respectively, for male FXS patients.

FIGS. 4A and 4B show the relation between FMRP as measured by assay and CGG repeat count and methylation load, respectively, for male and female FXS patients.

FIG. 5 shows the relation between methylation load and deviation IQ in male (blue) and female (red) FXS patients; and

FIGS. 6A and 6B show the difference between assay-detected or no assay-detected FMRP and CGG repeat count and methylation load, respectively, in fully methylated male patients.

FIGS. 7A and 7B show the FMR1 CCG repeat length is negatively correlated with FMRP levels.

FIG. 8 shows that ONT CGG repeat length negatively correlates with IQ in FXS.

Additional aspects of the invention will be more fully described in the following description.

### DETAILED DESCRIPTION

The methods of the invention for evaluating FXS disease phenotypes, and/or methylation and CGG extent, are suitable for use in assessing expected long-term disability and in providing an opportunity for early intervention and/or accommodation and support.

The methods prepare DNA from patients with Fragile X Syndrome using a CAS9 mediated enrichment of the CGG repeat region in the UTR of the FMR1 gene. Thus, the first step is to isolate high molecular weight DNA from a whole blood sample from the patient. Various techniques are known in the art and may be employed to provide the isolated high molecular weight DNA sample. One example of high molecular weight DNA extraction methods include the use of PacBio's Nanobind® kits, namely The Pan DNA kit and the CBB kit. Please see PacBio, Extracting HMW DNA from human whole blood using Nanobind® kits, Procedure & checklist, July 2022, revised August 2024, pp. 1-10, which is incorporated herein in its entirety. The PanDNA® kit contains three (3) wash buffers (CW1, CW2 and, PW1) to extract various sample types. The CBB kit only contains 2 wash buffers (CW1 and CW2). Buffers CW1, CW2, and PW1 are supplied as concentrates. CW1 and CW2 are used with a 60% final ethanol concentration. PW1 is used with a 70% final ethanol concentration.

In an Eppendorf tube, including Eppendorf, Protein LoBind® tubes, the Proteinase K is added. The whole blood sample is then added to the tube with the Proteinase K. RNase A is then added. The tube is then centrifuged and liquid is removed from the tube. Buffer BL3 is then added to the tube and the tube is again centrifuged. The tube is incubated for approximately 10 minutes in a thermomixer, heat block or water bath. After incubation the tube is centrifuged again. A Nanobind® disk is added to the tube and subsequently isopropanol is added. The contents are mixed. The tube is then centrifuged and the supernatant is removed. Buffer CW1 is added and mixed followed by removal of the supernatant. Buffer CW2 is added and mixed followed by removal of the supernatant. The step with Buffer CW2 can be repeated. Afterward the tube is centrifuged and any residual liquid is removed. This step can be repeated. Next, Buffer LTE is added to the tube and the tube is incubated for approximately 10 minutes. The DNA is then collected from the tube. See PacBio's Extracting HMW DNA from human whole blood using Nanobind® kits reference.

Another example of a high molecular weight DNA extract method includes the use of the Qiagen Flexigene DNA extraction kit. This kit includes a lysis buffer (Buffer FG1), a denaturation buffer (Buffer FG2) and a hydration buffer (Buffer FG3) along with a protease. To a whole blood sample the lysis buffer is added. The volume of the buffer depends upon the starting volume of DNA. The buffer may be added in a volume of 7.5 ml for 3 ml starting volume, 5 ml for 2 ml and 2.5 ml for 1 ml. The mixture is subjected to centrifugation to form a pellet of the cell nuclei and mitochondria. The pellet of cell nuclei and mitochondria is resuspended in the denaturation buffer with the protease. The denaturation buffer may be added at a volume of about 1.5 ml, 1 ml and 0.5 ml for about 3 ml, 2 ml and 1 ml of DNA starting volumes, respectively. This mixture is allowed to incubate. DNA is precipitated from this mixture by adding isopropanol in the same volume as for denaturation buffer (about 1.5 ml, 1 ml and 0.5 ml for about 3 ml, 2 ml and 1 ml of DNA starting volumes), centrifuged, washed with ethanol, in some embodiments 70% ethanol, and then dried. The DNA is then resuspended in the hydration buffer in a volume of between about 100 and about 300 μl depending upon the DNA starting volume and pellet size. The resuspended DNA is then ready for use in applications such as PCR, Southern blotting, restriction digestion, sequencing, and archiving.

The sample is then subjected to Cas9-assisted gene-targeted cleavage of regions of interest (e.g., the human FMR1 gene: NC_000023.11 (147911919 . . . 147951125 (assembly: GRCh38.p14 (GCF_000001405.40)))) in the 5′ UTR (untranslated region) of FMR1 gene to enrich the CGG regions with ligation of sequencing adapters to the cleaved regions of interest. Cas9-assisted enrichment is a process in which the Cas9 enzyme is combined with two specific cRNAs, (1) a structural RNA required for catalytic activity, and (2) custom-designed probes which are complementary to the sequence flanking the target region, to form a ribonucleoprotein complex using, for example the ligation sequencing gDNA-Cas9 enrichment kit from ONT (SQK-CS9109). Genomic DNA in the isolated sample is first dephosphorylated to block the strand ends by preventing ligation to the sequencing adapters. Ribonucleoprotein complexes, such as the Cas9 with FMR1 gene region, for each target region are then added and bind to the DNA sequence dictated by the RNA probes, for example, for FMR1 5′UTR: FMR1_D2 ATCACGATCCCAATCTTCTCGTTTTAGAGCTATGCTA; FMR1_U2: TTTAGGCTTGAGCAACGAACGTTTTAGAGCTAT, flanking each region of interest, and enabling Cas9 to cut the DNA. This releases phosphorylated ends, allowing the attachment of sequencing adapters to the exposed target molecules. The depletion of unwanted, unphosphorylated DNA frees up sequencing time for regions of interest, producing tens-to hundreds-fold enrichment.

By enriching native DNA, this provides an effective method of targeting very large regions, particularly regions for which PCR-amplification is not desired. PCR is not as effective for sequencing regions that are GC rich or highly repetitive as it can be difficult or even impossible to amplify, and PCR also removes base modifications preventing their analysis. Accordingly, the ability to conduct the present methods in the absence of, i.e., free from, a PCR step, is advantageous.

Any desired number of reads of regions of interest, i.e., fragments of DNA in the CGG repeat region, may be generated and sequenced. In one embodiment, the enriching step produces at least about 20 regions of interest. In another embodiment, the enriching step produces at least about 50 regions of interest. Any number of regions is possible, on the FMR1 gene, including the 5′ or 3′UTR of coding regions.

Sequencing may be conducted using any technique known in the art that allows detection of methylation in the resulting reads, such as bisulfite sequencing, single molecule real-time (SMRT) sequencing, and nanopore sequencing. Bisulfite sequencing refers to the conversion of unmethylated cytosines to uracils with bisulfite treatment. See Sigurpalsdottir et al., “A comparison of methods for detecting DNA methylation from long-read sequencing of Human genomes”, Genome Biology, Vol. 25, No. 69, 2024, pp. 1-21, which is incorporated herein in its entirety. In this approach, methylated CGGs remain as cytosines. PCR amplification of the DNA converts uracils to thymine. CGG methylation to be inferred indirectly from the sequenced DNA. Id.

SMRT sequencing utilizes hairpin adapters to attach to the DNA fragments thereby creating a single-stranded circular template that can be sequenced. Id. SMRT sequencing can distinguish modified bases from unmodified bases by measuring the time it takes to incorporate the next base during the DNA synthesis process as modified bases alter the kinetics of this process. Id.

In a specific embodiment, nanopore sequencing is employed, and more particularly, nanopore long read sequencing is employed, suitably using the Oxford nanopore long read sequencing platform. Id. This technique allows determination of a cumulative number of CGG repeats in the regions of interest, referred to herein as the CGG count, and a cumulative number of methylated CGG repeats in the regions of interest, referred to herein as the methylation load. The method is described in detail in Simpson J T, Workman R E, Zuzarte P C, David M, Dursi L J, Timp W. Detecting DNA cytosine methylation using nanopore sequencing. Nat Methods. 2017; 14 (4): 407-10. PubMed PMID: 28218898, which is incorporated herein in its entirety. Briefly, in addition to determining nucleic acid sequence, the electrolytic currents generated by guiding the methylated DNA through Nanopores can be used to predict methylation status of cytosines.

The methylation load and CGG count can be related to particular Fragile X Syndrome phenotypes, which facilitates decision making for care strategies at an early point in the patient development. As will be discussed below, the methylation load can be correlated to deviation IQ, currently the gold standard for assessing Fragile X Syndrome phenotype.

Thus, according to the invention, methylation data may be used to determine a CGG repeat region methylation score, i.e., methylation load, by continuous measure of CGG repeat region methylation. In demonstrating the effectiveness of the inventive methods, this score shows correlation with FMRP expression and clinical phenotype in the same patients undergoing the inventive methods, confirming the correlation between the novel methylation testing output and the FMRP expression and clinical phenotype of the patients. Those skilled in the art will appreciate that the relation between methylation load and CGG count and particular FXS phenotype(s) may be analyzed in a variety of ways, for example, as set forth in the Figures herein and in additional, for example, non-linear manners, and, optionally, in conjunction with other parameters including, but not limited to, CGG and/or methylation location, FMRP measurement, and the like.

In specific embodiments, the inventive methods may be combined with FMRP measurement, i.e., the methods further comprise determining an amount of FMRP in the whole blood sample from the subject. FMRP can be measured by known methods in the art, including but not limited to immunoassays, such as ELISA and Luminex, as well as time-resolved fluorescence resonance energy transfer (TR-FRET). An optimized Luminex method described in Boggs et al., 2022 (Boggs A E, Schmitt L M, McLane R D, Adayev T, LaFauci G, Horn P S, Dominick K C, Gross C, Erickson C A. Optimization, validation and initial clinical implications of a Luminex-based immunoassay for the quantification of Fragile X Protein from dried blood spots. Scientific reports. 2022; 12 (1): 5617. PubMed PMID: 35379866; PubMed Central PMCID: PMC8980090), which is incorporated herein in its entirety, will be used, in which FMRP will be eluted from blood spots and blood cards, and assayed using two specific antibodies combined with a standard curve with recombinant FMRP and detected using a bead-based method. Advantageously, the relating step may then comprise relating the cumulative number of CGG repeats, the cumulative number of methylated CGG repeats, and the determined amount of FMRP to a clinical phenotype of Fragile X Syndrome in the subject.

In further embodiments, the methods may further comprise determining the CGG repeat location and the location of methylation of CGG repeats within the FMR1 gene. The CGG repeats may all be in the 5′ UTR of FMR1, but methylation can occur anywhere in this CGG repeat region. It is believed that methylation closer to the ATG translation start could be more detrimental because the methylation could not only interfere with transcription of the FMR1 mRNA but also the translation of the mRNA into protein.

Any of the methods of the invention may also further include a step of providing therapy or care to the subject based on the related clinical phenotype of Fragile X Syndrome. Advantageously, such therapy or care can be provided prior to or early in a patient's disability manifestation.

While subjects that fall into the normal and intermediate or grey zones may not require a specific treatment, monitoring subjects in the intermediate zone is advisable as the subject could develop further mutations as they age leading to other conditions or ailments. With regard to premutation carriers, there is an increased risk of developing health issues such as Fragile X-associated tremor/ataxia syndrome (FXTAS) in men, and Fragile X-associated primary ovarian insufficiency (FXPOI) in women. For those subjects that develop FXTAS, treatments include supportive care, physical therapy, medication to manage tremors, ataxia and balance issues. Such medications include primidone, memantine, eldepryl, propranolol, carbidopa, levodopa, topiramate, sotalol, atenolol, pramipexole, alprazolam, botulinum toxin, levetiracetam, clonazepam, clozaline, nadolol, nimodipine, beta-blockers, dopaminergic therapy, and benzodiazepines.

For those subjects that develop FXPOI, treatments include hormone replacement therapy (HRT), such as estrogen and other hormones that are low or missing in the subject, and fertility treatments, such as assisted reproductive technologies, including in vitro fertilization (IVF).

For those subjects that possess the full mutation, the subject may suffer from Fragile X syndrome, including intellectual disability, autism spectrum disorder and other developmental disorders. There is no cure for Fragile X syndrome and available treatments only tackle associated symptoms. Treatments for those with the full mutation often integrate a multidisciplinary approach, including but not limited to, one or more therapies, medication, and behavioral strategies. Therapies that can be employed include speech and language therapy, physical therapy, occupational therapy, behavioral therapy, sensory integration training, behavior modification programs, psychotherapy, and group therapy, including social skills-oriented group therapy. Medications that may be helpful for those with Fragile X Syndrome include antidepressants, stimulants, sympatholytics, antipsychotics, gabapentin, methylphenidate, clonidine, propranolol, pindolol, carbamazepine, valproic acid, clonazepam, risperidone, lithium, anticonvulsants, selective serotonin reuptake inhibitors (SSRIs), buspirone, beta blockers, melatonin, minocycline, cannabidiol (CBD), and medications for synaptic dysfunction. There are no specific treatments yet targeting intellectual disability and there are no approved medication treatments for FXS.

The methods described to determine CGG repeat number and methylation will provide a more accurate and precise molecular phenotype of patients with FXS than conventional methods. They may allow a better disease prognosis and provide rationale for individualized treatment strategies in FXS. Additionally, this level of molecular genetic specificity will enable enhanced subgroup determination within clinical trials to determine which specific molecular genetic features correlate with treatment response.

The methods may comprise, consist of, or consist essentially of the elements of the methods as described herein, as well as any additional or optional element described herein or otherwise useful in methods for determining methylation status and CGG repeat extent in an untranslated region (UTR) region of Fragile X messenger riboprotein gene 1 (FMR1) in a subject, or in methods for evaluating a subject for Fragile X Syndrome.

### EXAMPLES

The following procedure is useful for practicing the inventive methods.

### Cas9-Assisted FMR1 Gene Isolation:

High-molecular weight DNA is isolated from whole blood using the Qiagen Flexigene DNA extraction kit (cat no. 51206), which reliably produces DNA that meets or exceeds ONT quality requirements, and provides an isolated sample for the Cas9-assisted gene-targeting of the FMR1 gene. The Cas9-assisted gene enrichment step (nCATS) uses targeted DNA cleavage and ligation of sequencing adapters allowing for sequencing of specific DNA fragments as described in Giesselmann et al., “Analysis of short tandem repeat expansions and their methylation state with nanopore sequencing”, Nature Biotechnology, 2019, Vol. 37, No. 12, pp. 1478-1481. doi: 10.1038/s41587-019-0293-x, which is incorporated herein in its entirety. The cRNAs are designed using the online tool CHOPCHOP and work reliably. The ONT SQK-LSK109 kit is used to prepare the enriched FMR1 DNA fragments for sequencing. This kit includes L fragment buffer, ligation buffer, sequencing buffer, a DNA control strand, an adapter mix, S fragment buffer, elution buffer and loading beads.

### Nanopore Sequencing and Analysis:

Nanopore sequencing is performed with the Oxford Nanopore MinION Mk1C. Preliminary analyses have been conducted using METEORE program's Nanopolish pipeline to detect CpG methylation and WarpSTR to detect number of trinucleotide repeats. Methylation load (e.g., total methylation in the repeat region) is calculated to quantify methylation, but will more refined measurements, for example, location of methylations, may be included as needed. The resulting data indicates that the more methylation, the less FMRP and the lower the IQ. It is believed that the more severe phenotype correlates with more methylation.

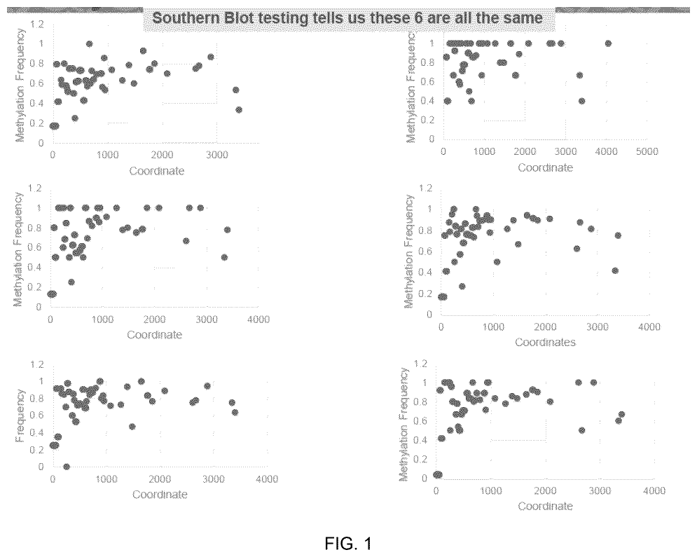

FIG. 1 shows the shortcomings of the conventional Southern Blot diagnosis of six “fully methylated” male patients. The six patients which were the subject of these results had differing phenotypes of Fragile X Syndrome, which could not have been predicted from the Southern Blot results.

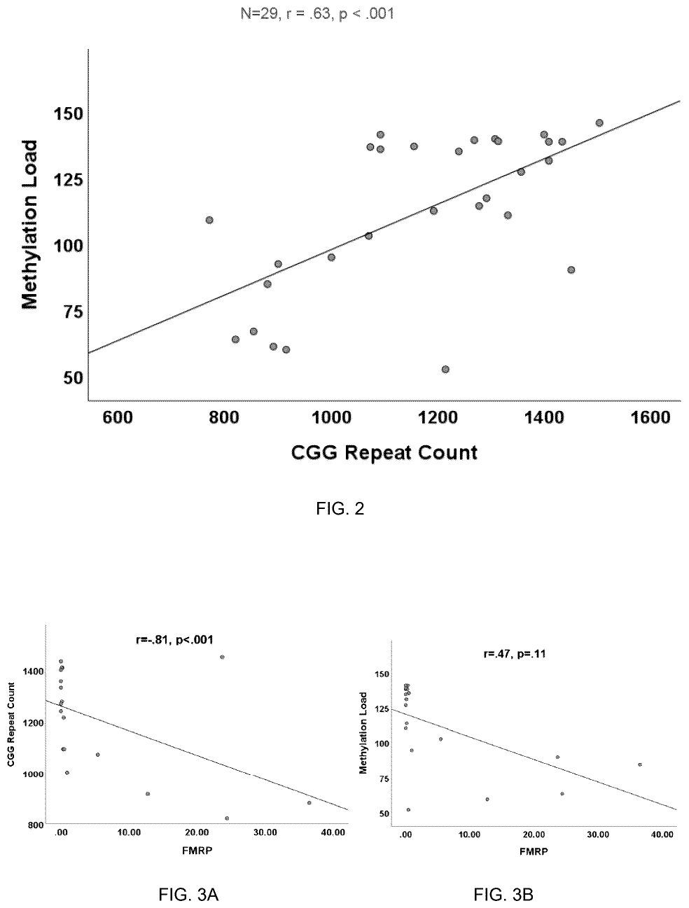

FIG. 2 shows the relation between CGG count and methylation load. Generally, the higher CGG repeat count, the higher methylation load.

FIGS. 3A and 3B show the relation between FMRP as measured by assay and CGG repeat count and methylation load, respectively, for a group of participants comprising male FXS patients. Multiple patients of varying CGG repeat count and methylation load had essentially zero FMRP detection. Generally, higher FMRP correlated with lower CGG count and methylation load.

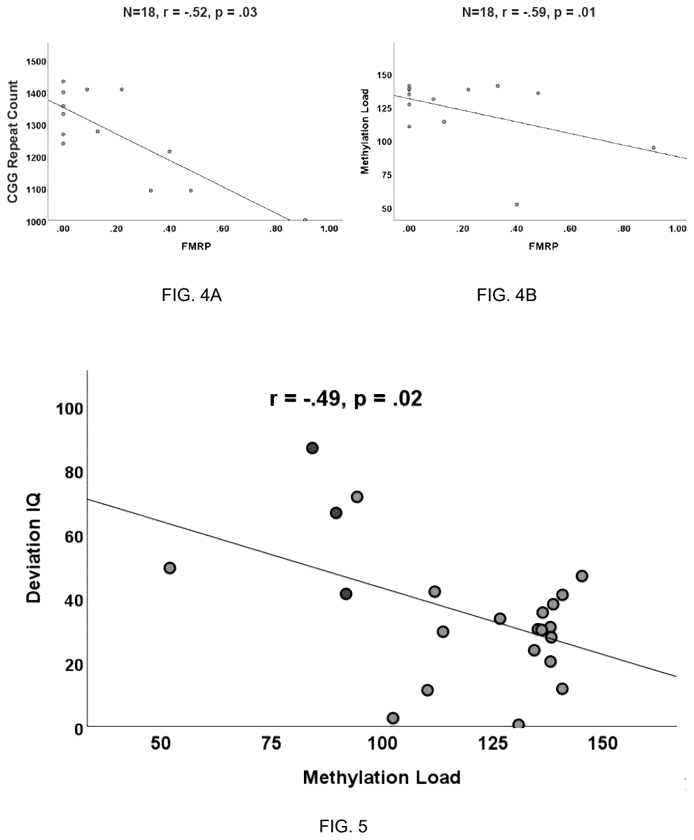

FIGS. 4A and 4B show the relation between FMRP as measured by assay and CGG repeat count and methylation load, respectively, for a group of male and female FXS patients. As in FIGS. 3A and 3B, multiple patients of varying CGG repeat count and methylation load had essentially zero FMRP detection. Generally, there was a higher correlation between higher FMRP and lower CGG count and methylation load.

FIG. 5 shows the relation between methylation load and deviation IQ for a group of male and female FXS patients, evidencing that the higher methylation load correlates with a lower deviation IQ, and thus evidencing an important improvement provided by the inventive methods for using methylation load to predict IQ in Fragile X Syndrome patients.

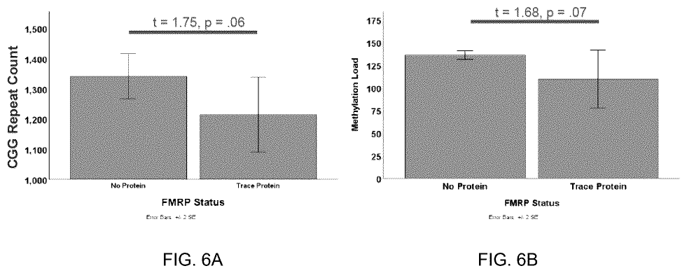

FIGS. 6A and 6B show the difference between assay-detected or no assay-detected FMRP and CGG repeat count and methylation load, respectively, in fully methylated male patients. Generally, higher CGG count and higher methylation load is found in patients with no detectable FMRP.

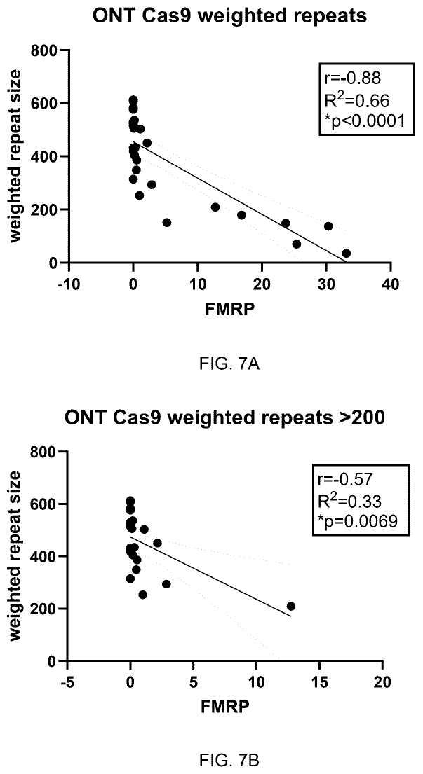

Further, FIGS. 7A and 7B show that weighted average repeat lengths (based frequency of reads with a certain repeat number per patient) determined by ONT/Cas9 sequencing negatively correlates with FMRP levels in a cohort of FXS patients and typically developing controls. Shown in FIG. 7B are cases with repeat lengths>200, traditionally categorized as “full mutation” and “no protein expression”. The negative correlation suggests that even when the repeat length is above the “full mutation” threshold, there are differences in FMRP expression which could affect IQ.

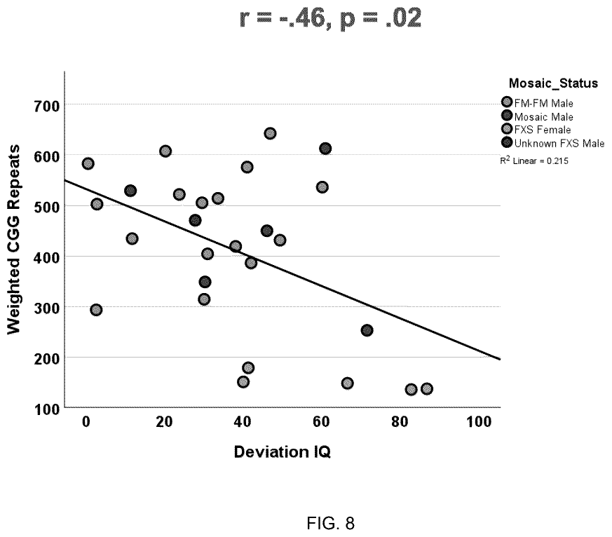

Finally, FIG. 8 shows that ONT CGG repeat length negatively correlates with IQ in FXS. This suggests that specific repeat length correlates with the severity of developmental impairment in FXS. Shown are weighted repeat lengths plotted against the deviation IQ. The weighted repeat length of an individual is calculated from the proportion of reads assigned to a specific CGG repeat length: e.g., if two different repeat lengths are detected, with 20 reads assigned to repeat length 1 (RL1, 400 repeats) and 30 assigned to repeat length 2 (RL2, 600 repeats) then the weighted CGG repeat length (WRL) is WRL=20*400/(20+30)+30*600/(20+30)=520). IQ is negatively correlated with weighted CGG repeat length in male and female patients with FXS.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for determining methylation status and CGG repeat extent in an untranslated region (UTR) of Fragile X messenger riboprotein gene 1 (FMR1) in a subject, comprising:
(a) isolating high molecular weight DNA from a whole blood sample from the subject;
(b) enriching the isolated high molecular weight DNA for the UTR of FMR1 with Cas9-assisted gene-targeted cleavage of regions of interest in the UTR of FMR1 gene and ligation of sequencing adapters to the cleaved regions of interest; and
(c) sequencing the regions of interest to determine a cumulative number of methylated CGG repeats in the regions of interest and a cumulative number of CGG repeats in the regions of interest.

2. The method of claim 1, wherein the sequencing step employs a nanopore sequencing platform.

3. The method of claim 1, further comprising providing therapy to the subject based on the cumulative number of CGG repeats in the regions of interest and the cumulative number of methylated CGG repeats in the regions of interest.

4. The method of claim 1, further comprising determining an amount of Fragile X messenger ribonucleoprotein (FMRP) in the whole blood sample from the subject.

5. The method of claim 4, further comprising providing therapy to the subject based on the cumulative number of CGG repeats in the regions of interest, the cumulative number of methylated CGG repeats in the regions of interest, and the determined amount of FMRP.

6. The method of claim 1, further comprising determining FMR1 gene locations of the CGG repeats in the regions of interest and/or the methylated CGG repeats in the regions of interest.

7. A method for evaluating a subject for Fragile X Syndrome, comprising:
(a) isolating high molecular weight DNA from a whole blood sample from the subject;
(b) enriching the isolated high molecular weight DNA for an untranslated region (UTR) region of Fragile X messenger riboprotein gene 1 (FMR1) with Cas9-assisted gene-targeted cleavage of regions of interest in the UTR of FMR1 gene and ligation of sequencing adapters to the cleaved regions of interest;
(c) sequencing the regions of interest to determine a cumulative number of methylated CGG repeats in the regions of interest and a cumulative number of CGG repeats in the regions of interest;
(d) relating the cumulative number of CGG repeats and the cumulative number of methylated CGG repeats to a clinical phenotype of Fragile X Syndrome in the subject; and
(e) personalize a therapy for the subject based on the clinical phenotype of Fragile X Syndrome.

8. The method of claim 7, wherein the sequencing step employs a nanopore sequencing platform.

9. The method of claim 7, wherein the enriching step produces at least about 20 regions of interest.

10. The method of claim 7, further comprising determining an amount of Fragile X messenger ribonucleoprotein (FMRP) and/or FMR1 mRNA in the whole blood sample from the subject.

11. The method of claim 10, wherein the relating step comprises relating the cumulative number of CGG repeats, the cumulative number of methylated CGG repeats, and the determined amount of FMRP to a clinical phenotype of Fragile X Syndrome in the subject.

12. The method of claim 7, further comprising providing therapy to the subject based on the related clinical phenotype of Fragile X Syndrome.

13. The method of claim 7, further comprising determining FMR1 gene locations of the CGG repeats in the regions of interest and/or the methylated CGG repeats in the regions of interest.

14. The method of claim 1, conducted in the absence of PCR amplification.

15. The method of claim 7, conducted in the absence of PCR amplification.
